(19) United States
(12) Patent Application Publication
(10) Pub. No.: US 2011/0280894 A1
(43) Pub. Date: Nov. 17, 2011

(54) HER2/NEU SPECIFIC T CELL RECEPTORS

(76) Inventors: Angela Krackhardt, Munchen (DE); Luise Weigand, Munchen (DE); Xiaoling Liang, Munchen (DE)

(21) Appl. No.: 13/056,827

(22) PCT Filed: Jul. 31, 2009
(86) PCT No.: PCT/EP09/59953

§ 371 Date: Jul. 29, 2011

(30) Foreign Application Priority Data
Jul. 31, 2008 (EP) 08161539.5

(51) Int. Cl. A61K 39/00; C12N 5/0783; C12N 5/10; A61P 37/04; A61K 35/12; C07K 14/725; C07H 21/04; A61P 35/00; C12N 15/63; A61K 35/14

(52) U.S. Cl. 424/184.1; 435/320.1; 435/325; 435/372; 424/93.21; 424/93.7; 435/375; 530/324; 536/23.5

(57) ABSTRACT

The present invention is directed to T cell receptors (TCR) recognizing antigenic peptides derived from Her2/neu, in particular peptide 369, and being capable of inducing peptide specific killing of a target cell overexpressing HER2/neu. The present invention is further directed to an antigen specific T cell, comprising said TCR, to a nucleic acid coding for said TCR and to the use of the antigen specific T cells for the manufacture of a medicament for the treatment of malignancies characterized by overexpression of HER2/neu. The present invention is further disclosing a method of generating antigen specific T cells.

### BACKGROUND OF THE INVENTION

The present invention is directed to T cell receptors (TCR) recognizing antigenic peptides derived from Her2/neu, in particular peptide 369, and being capable of inducing peptide specific killing of a target cell overexpressing HER2/neu. The present invention is further directed to an antigen specific T cell, comprising said TCR, to a nucleic acid coding for said TCR and to the use of the antigen specific T cells for the manufacture of a medicament for the treatment of malignancies characterized by overexpression of HER2/neu. The present invention is further disclosing a method of generating antigen specific T cells.

Allogeneic hematopoietic stem cell transplantation (SCT) is an effective therapy for hematologic malignancies with curative scope. Although there are encouraging data using allogeneic stem cell transplantation with reduced intensity conditioning regimens in several solid malignancies, this approach has been much less successful than in hematologic disorders (1, 2). Allogeneic stem cell transplantation has been applied with some success in renal-cell carcinoma (3). Complete remissions after adoptive T-cell transfer have also been observed in metastatic breast cancer but are associated with graft-versus-host-disease (GνHD) (4, 5). As GνHD is associated with a high morbidity and mortality particularly reducing therapeutic outcome, an important future goal is therefore the improvement of specificity of antitumor immune responses by reducing GνHD and increasing Graft-versus-tumor (GνT) effects.

Allorestricted T cells with specificity for epitopes derived from tumor-associated antigens (TAA) may represent a therapeutic approach to reduce the risk of alloreactivity (6). However, such allorestricted peptide-specific T cells may display high avidity towards MHC-presented TAA, since these MHC/peptide combinations were not present during thymic negative selection. Moreover, they can be isolated by MHC/peptide-multimers and cloned by limiting dilution to identify the specific TCR responsible for tumor-selective killing (7). The isolation of a TCR with defined specificity for TAA facilitates genetic TCR transfer into PBMC (8-11), allowing expansion of tumor-specific T cells. Using such allorestricted peptide-specific TCR tumor-specific effects may be achieved while significantly reducing the risk of GνHD (12).

The HER2/neu protein is an intensively investigated tumor-associated antigen (TAA) and a member of the tyrosine kinase family of growth factor receptors (13, 14). HER2/neu is a transmembrane glycoprotein which increases receptor tyrosine phosphorylation correlating with cellular transformation in a dose-dependent manner (15). Overexpression of HER2/neu, with subsequent constitutive kinase activation, is found in approximately 20-30% of human breast cancers and is associated with reduced disease-free and overall survival (16). HER2/neu can be used to target breast cancer cells and, additionally, a wide range of tumors of different origins that aberrantly express HER2/neu (4). Monoclonal antibodies (Trastuzumab) against HER2/neu have been shown to be effective in HER2/neu-positive breast cancers (17). However, the majority of metastatic breast cancer patients initially responding to Trastuzumab, show disease progression within one year (18). In addition, immunogenic peptides derived from HER2/neu have been defined and T cells with antitumor activity have been selected in vitro (12, 19, 20). However, results of vaccination studies with HER2/neu-derived peptides were ambivalent (21-24). In one single study, autologous HER2/neu-specific cytotoxic T cells have been used for adoptive T-cell transfer in one single patient with HER2/neu-overexpressing metastasizing breast cancer, demonstrating some effectivity in the bone marrow but not in solid metastasis (25). It has not been clear whether this limited success reflects suboptimal vaccination or T cell generation strategies which elicited only low-avidity T cell receptors (TCR).

### SUMMARY OF THE INVENTION

Therefore, it is one object of the present invention to provide a new and improved approach for immunotherapies of tumors characterized by an overexpression of Her2/neu, in particular of breast cancer, wherein the disadvantages of the conventional therapies may be avoided.

It is a further object of the present invention to generate allorestrictive T cells that bear TCR that have the capacity to recognize their MHC-peptide ligands on tumor cells. Furthermore, it is an object of the invention to provide a T cell based pharmaceutical composition that can be used for treating a patient suffering from tumors characterized by an overexpression of Her2/neu without a risk of graft-versus-host-disease (GVHD).

These objects are achieved by the subject-matter of the independent claims. Preferred embodiments are set forth in the dependent claims.

The inventors have generated allorestricted peptide-specific T cells with specificity against defined peptides derived from the tumor associated antigen Her2/neu derived peptide 369 (see SEQ ID NO:1).

Allorestricted T-cell lines and clones with specificity for the HER2/neu-derived peptide 369 were generated using peptide-pulsed T2 cells. Clones could be identified demonstrating high peptide specificity and tumor reactivity in screening assays while alloreactivity was low. The specificity of these T-cell clones could be transferred on PBMC by retroviral TCR transfer and the TCR-transduced PBMC recognized endogenously processed HER2/neu antigen since HLA-A2⁺ K562 cells transfected with HER2/neu but not mock-transfected HLA-A2⁺ K562 cells were recognized. Moreover, these TCR-transduced PBMC recognized different HER2/neu overexpressing tumor-cell lines. These TCR's are therefore a highly promising tool for the development of specific adoptive T-cell therapies to treat HER2/neu overexpressing tumors.

As already mentioned above, adoptive T-cell transfer has been shown to be highly effective using ex vivo expanded tumor-infiltrating lymphocytes (TIL) in patients with metastatic melanoma. This therapeutic approach has also been shown to be feasible in breast carcinoma and complete remissions after adoptive transfer have been observed in an allogeneic setting. However, this approach using adoptive T cell transfer has been associated with high morbidity due to GνHD.

The inventors tested if allorestricted T cells with specificity for the HER2/neu-derived peptide 369 with low crossreactivity may be generated and therefore reduce risk of GνHD.

The inventors as a result generated HER2/neu-specific allorestricted T cells in different stimulation conditions using peptide-pulsed T2 cells for stimulation. These different conditions included high and low peptide concentrations as well as single or repeated stimulation.

Using two different doses of peptide (10 μM versus 0.1 μM) and either single or repeated stimulation, the inventors mainly identified two characteristic groups of specific T-cell clones dominating the allorestricted HER2/neu-specific repertoire. One group (pattern 1) was represented by T cells with preferential recognition of HER2/neu 369 and enhanced tumor reactivity, but these T cells had partial unspecific reactivity against an unrelated peptide. The second group (pattern 2) was represented by T cells highly specific for the HER2/neu-derived peptide 369. However, these T cells showed no reactivity against tumor cells.

Only one out of 97 T-cell clones showed the favourable properties of high peptide specificity and tumor reactivity in the screening assay. Although experiments presented here consider the allo-HLA-A2-restricted HER2/neu-specific T-cell repertoire of only one healthy HLA-A2⁻ donor, the fact that this T-cell clone with high peptide specificity and tumor reactivity was generated by single antigen exposure to a low antigen concentration suggest that such T cells are rare and quickly deleted after repeated stimulation or stimulation with higher antigen concentrations or are overgrown by less avid T cells.

In the present invention most T-cell clones, particularly T-cell clone D1 (HER2-1), did not survive over a longer period in vitro, impeding extensive testing of this clone. Thus, the TCR usage was investigated for selected clones in order to facilitate genetic transfer of TCR chains into PBMC. TCR D1 (HER2-1), G3 (HER2-2), 96 (HER2-3) and E1 (HER2-4) representing the TCR of highly HER2/neu-peptide-specific T-cell clones, were selected for TCR transfer studies and both TCR could be transduced as single genes into PBMC resulting in stable HER2/neu (369) multimer-positive cells with peptide-specific functional activity. Transfer of TCR D1 (HER2-1) in PBMC resulted not only in specific recognition of peptide-pulsed T2 cells but also recognition of tumor cells that present endogenously processed HER2/neu and presented it in the HLA-A2 context. Moreover, TCR D1 (HER2-1)-transduced PBMC did not show notably peptide crossreactivity or alloreactivity.

In addition, different modifications, such as murinization of the TCR constant regions, the use of synthetic genes with optimized codon usage and introduction of additional disulfide bonds may further enhance TCR-D1 (HER2-1) expression and function of transduced PBMC which need to be investigated prior to any clinical trials.

In conclusion, the results presented herein are based on an investigation of the HLA-A2-allorestricted TCR repertoire against HER2/neu (369) using peptide-pulsed T2 cells in vitro. The TCR usage of various HER2/neu-reactive T cell clones with high or low crossreactivity and different avidities from the allo-HLA-A2-restricted T cell repertoire of a healthy HLA-A2⁻ individual was investigated. The corresponding TCR's are a highly promising tool for the development of adoptive T-cell therapies in patients with HER2/neu-overexpressing cancer.

### DETAILED DESCRIPTION OF THE INVENTION

According to a first aspect, the invention provides a T cell receptor (TCR) recognizing HER2/neu derived peptide 369 and capable of inducing peptide specific killing of a target cell overexpressing HER2/neu, wherein the TCR specifically recognizes the peptide of SEQ ID NO: 1. The peptide according to SEQ ID NO: 1 corresponds to peptide 369.

The term “TCR” as used in the present invention has the common meaning, which usually is attributed to that term in the pertinent field of technology. Thus, a rearranged T cell receptor (TCR) comprises a complex of two chains (α-chain and β-chain) containing a CDR3-region of rearranged TCR VDJ genes mainly involved in the recognition of antigenic determinants (epitopes) represented in the MHC context. More detailed information can be found in “Immunobiology, the immune system in health and disease”, Charles A. Janeway, et al, 5 ed. 2001 and other standard literature.

An “antigenic peptide” as used herein is defined as comprising at least one antigenic determinant, i.e. an epitope. The latter is a part of a macromolecule that is being recognized by the immune system, in the present case specifically by cytotoxic T cells.

Accordingly, the TCR of the present invention specifically recognizes one the peptide of SEQ ID NO: 1 and/or peptides/proteins containing same.

According to a preferred embodiment, the TCR of the present invention contains or consists of one of the amino acids of the TCR alpha chains of SEQ ID NO: 2-14 and/or one of the amino acids of the TCR beta chains of SEQ ID NO: 15-24. It is noted that this means that the alpha chains or beta chains may be used alone or in combination with each other.

It is further noted that all of the above mentioned sequences have a C-terminal Gly residue. This residue is a conserved residue of T cell receptors without any effect on the specificity of the TCR. Therefore, the present invention is also directed to those sequences having Gly removed at the C terminus.

It surprisingly turned out that the above indicated TCR's are showing high peptide specificity and tumor reactivity. More precisely, the TCR are allorestricted with specificity for the HER2/neu-derived peptide 369 and show only low crossreactivity and, thus, the risk of developing GvHD is considerably reduced.

In a preferred embodiment, the invention provides a TCR, which contains or consists of the amino acids of the TCR alpha chain of SEQ ID NO: 2. This TCR in the following is also termed D1 (HER2-1), which is by far the most promising TCR identified. PBMC transduced with the TCR D1 (HER2-1) show high specificity for the Her2/neu peptide 369 and low cross-reactivity. The crossreactivity of TCR D1 (HER2-1)-transduced PBMC against a panel of control peptides was tested. None of them was recognized by D1 (HER2-1)-transduced PBMC. Specificity of transduced TCRs for endogenously processed HER2/neu antigen could be further demonstrated using HLA-A2⁺ C1R cells transfected with HER2/neu. TCR-D1 (Her2-1) transduction of PBMC resulted in highly enhanced recognition and lysis of HER2-neu transfected HLA-A2⁻ C1R cells.

PBMC transduced with the HER2/neu-specific TCR D1 (Her2-1) further show tumor reactivity: PBMC transduced with TCR D1 (Her2-1) were observed as having reactivity against different tumor targets including SK-Mel 29 and MCF-7.

As mentioned above, the TCR of the present invention contains or consists of one of the amino acids of the TCR alpha chains of SEQ ID NO: 2-14 and/or one of the amino acids of the TCR beta chains of SEQ ID NO: 15-24. This means that the alpha chains or beta chains may be used alone or in any combination with each other.

The TCR alpha-chain of G3 (HER2-2) disclosed herein specifically recognized HER2₃₆₉ not only in combination with the original β-chain but also with other beta-chains of the same variable family deriving from TCR with diverse specificities. Pairing with one beta-chain derived from another HER2₃₆₉-specific TCR potentiated the chimeric TCR in regard to functional avidity, CD8 independency and tumor reactivity. Although the frequency of such TCR single chains with dominant peptide recognition is currently unknown, they represent interesting tools for TCR optimization resulting in enhanced functionality when paired to novel partner chains.

By investigating the potential pairing of single TCR chains, the inventors were able to demonstrate that the specificity of a TCR can be mainly defined by a single TCR alpha-chain, but pairing with alternative beta-chains influences peptide specific functional avidity, CD8 dependency and natural target recognition. Moreover, investigation of mixed chain chimeras revealed one alpha-chain derived of a HER2₃₆₉-specific TCR recognizing the specific peptide in combination with beta-chains derived from TCR with diverse specificities. Importantly, one novel combination of TCR alpha-beta-chains derived from two HER2₃₆₉-specific TCR primarily lacking tumor reactivity resulted in enhanced functional avidity, CD8 independency and tumor target recognition.

As such, repairing of the TCR alpha-chain according to SEQ ID NO: 5 with diverse beta-chains of the TRBV12 family results in enhanced HER2₃₆₉-specific functional avidity, CD8-independency and tumor reactivity. Combinations of the TCR alpha-chain according to SEQ ID NO: 5 with different beta-chains of the TRBV12 family displayed specific reactivity for T2 cells pulsed with HER2₃₆₉ in a dose-dependent manner (Table 5). Whereas chimeric TCR combinations of G3α (HER2-2α) (=SEQ ID NO: 5) with D1 (HER2-1β) (=SEQ ID NO: 21) resulted in only marginally increased recognition of T2 cells pulsed with high concentrations of HER2₃₆₉, combinations of G3α (HER2-2α) with E1β (HER2-4β) (SEQ ID NO: 23) resulted in an increased functional avidity in peptide titration experiments when compared to the original chain combination. The combination of G3α HER2-2α and E1β (HER2-4β) demonstrated high peptide specificity for HER2₃₆₉ but also revealed increased reactivity in response to T2 cells pulsed with alternative peptides or unloaded T2 cells (Table 5). Moreover, the combination of HER2-2α and HER2-4β resulted in reactivity against diverse tumor cell lines (Table 5).

In parallel to the CD8-independent multimer staining of chimeric TCR combinations, mixed combinations of G3α (HER2-2α) with E1β (HER2-4β) expressed in CD8⁻ cells sorted after TCR-transfer by flow cytometry resulted in CD8-independent peptide recognition in a dose dependent manner demonstrating again a high functional avidity of these chimeric chain combinations (Table 6). Sorted CD8⁻ cells transduced with HER2-2α in combination with HER2-4β also demonstrated high peptide specificity, although background reactivity against T2 cells pulsed with alternative peptides was again noticed (Table 6). In addition, reactivity against selected tumor cell lines was observed (Table 6).

It is noted that the invention is not restricted to the precise amino acid sequences as defined herein, but also include variants of the sequences, for example deletions, insertions and/or substitutions in the sequence, which cause for so-called “silent” changes.

Preferably, such amino acid substitutions are the result of substitutions which substitute one amino acid with a similar amino acid with similar structural and/or chemical properties, i.e. conservative amino acid substitutions.

Amino acid substitutions can be performed on the basis of similarity in polarity, charges, solubility, hydrophobic, hydrophilic, and/or amphipathic (amphiphil) nature of the involved residues. Examples for hydrophobic amino acids are alanine, leucine, isoleucine, valine, proline, phenylalanine, tryptophan and methionine. Polar, neutral amino acids include glycine, serine, threonine, cysteine, thyrosine, asparagine and glutamine. Positively (basic) charged amino acids include arginine, lysine and histidine. And negatively charged amino acids include aspartic acid and glutamic acid.

The allowed degree of variation can be experimentally determined via methodically applied insertions, deletions or substitutions of amino acids in a peptide and testing the resulting variants for their biological activity as an epitope. In case of variation of the TCR-CDR3 region specificity and function of the modified TCR can be experimentally investigated by TCR expression in transduced cells or by purified TCRs analyzed with surface plasmon resonance (e.g. Biacore).

In a second aspect, the present invention provides an antigen specific T cell, comprising a TCR as defined above.

Said T cell preferably is a T cell with effector cell characteristics, more preferably a cytokine producing T cell, a cytotoxic T cell or regulatory T cell, preferably CD4+ or CD8+ T cells. Most preferably, the T cell is an autologous T cell. It may also be an allogeneic T cell.

In a third aspect, the invention provides a nucleic acid coding for a part of a TCR (CDR3-region) as defined above. Respective sequences are provided as SEQ ID NO: 25-47.

An additional aspect is directed to a vector or mRNA, which comprises the nucleic acid coding for said TCR. This vector is preferably an expression vector which contains a nucleic acid according to the invention and one or more regulatory nucleic acid sequences. Preferably, this vector is a plasmid or a retroviral vector.

The invention further comprises a cell, preferably a PBMC, which has been transformed with the vector as defined above. This can be done according to established methods.

In a still further aspect, the present invention provides a pharmaceutical composition, which comprises the T cells or cells as defined above and a pharmaceutically acceptable carrier.

Those active components of the present invention are preferably used in such a pharmaceutical composition in doses mixed with an acceptable carrier or carrier material, that the disease can be treated or at least alleviated. Such a composition can (in addition to the active component and the carrier) include filling material, salts, buffer, stabilizers, solubilizers and other materials, which are known state of the art.

The term “pharmaceutically acceptable” defines a non-toxic material, which does not interfere with effectiveness of the biological activity of the active component. The choice of the carrier is dependent on the application.

The pharmaceutical composition can contain additional components which enhance the activity of the active component or which supplement the treatment. Such additional components and/or factors can be part of the pharmaceutical composition to achieve synergistic effects or to minimize adverse or unwanted effects.

Techniques for the formulation or preparation and application/medication of active components of the present invention are published in “Remington's Pharmaceutical Sciences”, Mack Publishing Co., Easton, Pa., latest edition. An appropriate application is a parenteral application, for example intramuscular, subcutaneous, intramedular injections as well as intrathecal, direct intraventricular, intravenous, intranodal, intraperitoneal or intratumoral injections. The intravenous injection is the preferred treatment of a patient. According to a preferred embodiment, the pharmaceutical composition is an infusion or an injection or a vaccine.

According to a further aspect, the present invention is directed to the use of the antigen specific T cells or PBMCs as explained above for the treatment of tumors characterized by overexpression of HER2/neu, preferably breast cancer. Overexpression of Her2/neu also occurs in other cancer such as ovarian cancer and stomach cancer. Also those kinds of cancer may be treated with the composition of the present invention.

In a still further aspect, the invention provides a method of generating antigen specific allorestrictive T cells comprising the steps of

a) providing the HER2/neu derived antigenic peptide 369;

b) pulsing T2 cells with said peptide in a suitable concentration;

c) stimulating T cells with the peptide pulsed T2 cells;

d) selecting those T cells which are specific for the HER2/neu derived antigenic peptide.

The selection step d) is preferably performed by means of measuring the cytokine release of the T cells or other measures of T cell activation. For example, the activated T cells can be cloned as individual cells and following expansion, the T cell clones can be analyzed for their MHC-peptide specificity and those with the desired specificity can be selected for further use. Alternatively, soluble MHC-peptide ligands in various forms, such as tetramers, can be marked with a fluorescent label and incubated with the activated T cells. Those T cells bearing TCR that interact with the tetramers can then be detected by flow cytometry and sorted on the basis of their fluorescence. Furthermore, T cells can be stimulated for short periods of time with tumor cells to which they should react and their interferon gamma secretion detected by capture reagents, for example as published.

According to a preferred embodiment, the method of the invention further comprises the step of expanding the T cells selected in d) ex vivo.

The present invention in the following is illustrated by the Tables, Figures and Examples presented below, which in no way should be construed to be limiting the scope of the invention.

### BRIEF DESCRIPTION OF THE DRAWINGS

FIG. 1. Isolation of allorestricted HER2/neu-specific T cells. (A) HER2/neu-specific T cells in bulk cultures after two stimulations with HER2/neu (369)-pulsed (10 μM) T2 cells and (B) in sorted T-cell lines (one day after sorting) were quantified by flow cytometry using HER2/neu-specific HLA-A2-multimers. The numbers in the FACS plot represent percentage of cells in that region.

FIG. 2. Response pattern of isolated T-cell clones after T-cell cloning. Following HER2/neu (369)-peptide stimulation, FACS sorting and single cell cloning, T-cell clones were analyzed in a ⁵¹Cr-release screening assay at an excess E:T ratio for their reactivity against T2 cells pulsed with peptides derived from HER2/neu (369) or Flu as well as against the tumor-cell target SK-Mel 29. The 5 different reaction patterns displayed by individual isolated T-cell clones are shown.

FIG. 3. HER2₃₆₉-specific TCR are expressed after retroviral gene transfer.

(A) TCR α- and β-chain genes of the HER2₃₆₉-specific TCR HER2-1, HER2-2, HER2-3 and HER2-4 as well as the control TCR R6C12 with specificity for GP100₂₀₉ were retrovirally transduced into J76CD8 and analyzed by flow cytometry 4 days after transduction. Transduced cells were analyzed for specific TCR expression by staining with the specific multimer (thick line) as well as control multimers (thin line). (B) Similarly, single TCR chains of HER2₃₆₉-specific TCR and control TCR were retrovirally transduced into PBMC and stained with the specific multimer (upper panel) as well as the control multimer (lower panel) 10 days after transduction.

FIG. 4. HER2₃₆₉-specific TCR show peptide-specific function after retroviral gene transfer.

PBMC transduced with the HER2₃₆₉-specific TCR as well as the control TCR R6C12 were incubated 11 days after transduction for 24 hours with T2 cells pulsed with a range of titrated concentrations of HER2₃₆₉ (A), GP100₂₀₉ (B) or a panel of control peptides at 10 μM (C). Selected tumor cell lines were used as target cells for TCR-transduced PBMC (D). Tumor target cells were treated with IFN-γ (100 U/ml) 48 hours prior to the stimulation assay. Supernatants were analyzed by IFN-γ-ELISA. The numbers in brackets indicate the percentage of cells stained positive with the specific multimer. Standard deviations of triplicates are shown.

FIG. 5. PBMC transduced with modified TCR constructs show enhanced functions with preserved peptide specificity.

PBMC transduced with either single TCR chains (wildtype) or modified constructs (modified) were stimulated 11 days after transduction for 24 hours with target cells at E:T ratios of 5:1. Supernatants were then harvested and analyzed by IFN-γ-ELISA. The percentage of multimer-positive cells in the effector cell population is shown in the Supplement, Table SIII. Non-transduced PBMC as well as mock-transduced PBMC were used as controls. Standard deviations of triplicates are shown. (A) TCR transduced PBMC were tested against T2 cells pulsed with a range of titrated concentrations of specific peptide. HER2₃₆₉ was used for TCR HER2-1, HER2-2 and HER2-3; GP100₂₀₉ was used for TCR R6C12. (B) TCR transduced PBMC were tested against T2 cells pulsed with a set of alternative peptides at a concentration of 10⁻⁵ M. (C) TCR-transduced PBMC were tested against selected tumor cell lines. Tumor target cells were treated with IFN-γ (100 U/ml) 48 hours prior to the stimulation assay.

### EXAMPLES

PBMC from healthy donors were collected with donors' informed consent following the requirements of the local ethical board and the principles expressed in the Helsinki Declaration. PBMC subpopulations from healthy donors were isolated by negative or positive magnetic bead depletion (Invitrogen, Karlsruhe, Germany) and high purity was confirmed by flow cytometric analysis. The T2 cell line which is a somatic cell hybrid of human B- and T-lymphoblastoid cell lines (ATCC CRL-1992, Manassas, Va., USA) has been reported to be defective in transporter associated with antigen-processing (TAP) molecules and to be deficient in peptide presentation. Peptide-pulsed T2 cells were used for priming and restimulation of HLA-A2-negative T cells. The TCR-deficient T-cell line Jurkat76 (J76) and Jurkat76 transduced with CD8α (J76CD8) kindly provided by W. Uckert were used for TCR-transfer experiments. The following malignant cell lines were used as targets to test tumor reactivity and crossreactivity: HLA-A2-positive breast carcinoma cell lines MCF-7 (ATCC HTB-22) and MDA-MB 231 (CLS, Germany), the HLA-A2-negative ovarian cancer cell lines SKOV and SKOV transfected with HLA-A2 (SKOVtA2) (kindly provided by H. Bernhard), the HLA-A2-positive melanoma cell lines SK-Mel 29 and 624.38MEL (kindly provided by E. Noessner), wild type K562 (ATCC CCL-243), HLA-A2-positive 143 TK⁻ lung fibroblasts (kindly provided by R. Mocikat) and the human B-cell lines C1R untransfected and transfected with HLA-A*0201 (kindly provided by S. Stevanovic). CIR cells transfected with HLA-A*0201 and HER2 were kindly provided by J. Charo.

Peptides

The following peptides were used for pulsing of antigen-presenting cells: the HLA-A2-restricted HER2/neu-derived peptide 369 (KIFGSLAFL, SEQ ID NO: 1), the HLA-A2-restricted influenza matrix peptide MP58 (GILGFVFTL, SEQ ID NO: 48), the HLA-A2-restricted tyrosinase-derived peptide 369 (YMNGTMSQV, SEQ ID NO: 49), the Formin related protein in leukocytes (FMNL1)-derived HLA-A2-binding peptide PP2 (RLPERMTTL, SEQ ID NO: 50), and the HDAC6-derived peptide (RLAERMTTR, SEQ ID NO: 51) (26). Peptides were synthesized by standard fluorenylmethoxycarbonyl (Fmoc) synthesis (Biosyntan, Berlin, Germany). Purity was above 90% as determined by reverse phase high-performance liquid chromatography (RP-HPLC) and verified by mass spectrometry. Lyophilized peptides were dissolved in DMSO (Sigma) for 2 mM stock solutions.

Multimers and Antibodies

Multimers were synthesized as previously reported and used for detection and sorting of specific TCR (27-29). Specific multimers were used for the following peptides: A2-HER2/neu (369) and A2-Flu (MP58) (25). For selecting HER2/neu-specific T cells, multimer binding assays were performed essentially as previously described (30). The following antibodies were used to characterize PBMC-derived cells, primary tumor cells and malignant cell lines: anti-CD3-FITC (UCHT1, BD, Heidelberg, Germany), anti-CD4-FITC (RPA-T4, BD), anti-CD8-FITC (V5T-HIT8a, BD), anti-CD8-PE (RPA-T8, BD), anti-CD19-FITC and -PE (HIB19, BD), anti-CD14-PE (M5E2, BD), anti-CD56-PE (B159, BD), anti-HLA-A2-FITC (BB7.2, ATCC), anti-αβ-TCR-FITC (T10B9.1A-31, BD), anti-HER2 unlabeled (TA-1, Calbiochem), goat anti-mouse IgG-PE (Jackson ImmunoResearch).

CTLs

Cytotoxic T lymphocyte lines (CTL) were generated from PBMC using peptide-pulsed T2 cells for specific stimulation. T2 cells were pulsed with specific peptides (10 μM and 0.1 μM) and used for CTL priming at a stimulator:effector cell ratio of 1:10 and for restimulation at a stimulator:effector cell ratio of 1:100. Cytokines were added as follows: IL-2 (50 U/ml) (Chiron Vaccines International, Marburg, Germany), IL-7 (10 ng/ml) (Peprotech, London, UK) and IL-15 (10 ng/ml) (Peprotech). Peptide-specific T cells were detected by flow cytometry using PE-conjugated peptide-presenting HLA-A2⁺ multimers and sorted by a high performance cell sorter (MoFlo, Dako).

Sorted cells were cloned by limiting dilution and non-specifically restimulated every two weeks using pooled allogeneic irradiated PBMC together with anti-CD3 antibody (OKT3), IL-2, IL-7 and IL-15.

Functional Assays

For cytokine detection, effector and target cells were incubated at different effector-target (E:T) ratios for 24 h. Supernatants were collected and stored at −20° C. until analysis. The presence of IFNγ was analyzed by ELISA (BD) following the recommendations of the manufacturer.

Cytotoxic activity of CTLs was determined at different E:T ratios in a standard ⁵¹Cr-release assay, principally as previously described (31). T2 cells were ⁵¹Cr-labeled and loaded with peptide as indicated. T cells were added in different E:T ratios and cocultured for 4h at 37° C. CTL killing was calculated as the percentage of specific ⁵¹Cr release using the following equation: % specific lysis=[(sample release−spontaneous release)/(maximal release−spontaneous release)]×100.

TCR Analysis

PCR analysis of expressed TCR chains was performed as previously described (32). Total RNA from T-cell clones and lines was extracted according to the manufacturer's recommendation (Trizol reagent, Invitrogen). cDNA was synthesized using Superscript II reverse transcriptase (Invitrogen) and oligo dT primers. Subfamily-specific TCR-PCR was performed using 34 Vα and 37 Vβ primers followed by gel isolation (NucleoSpin, Macherey-Nagel, Düren, Germany) and direct DNA sequencing of the amplified products. The T-cell receptor nomenclature was used according to the WHO-IUIS nomenclature sub-committee on TCR designation (33).

Cloning of the HER2/Neu-Specific TCR

TCR cloning was performed as described (34). Shortly, the specific TCR α and β chain coding cDNA of clone D1 (Vα12.1 and Vβ8.1) and G3 (Vα10.1 and Vβ8.1) were amplified from isolated T-cell clones using variable chain-specific oligonucleotides containing a NotI restriction site: 5′Vα12.1-TAGCGGCCGCCACCATGCTGACTGCCAGCCTG (SEQ ID NO: 52), 5′Vα10.1-TAGCGGCCGCCACCATGGTCCTGAAATTCTCC (SEQ ID NO: 53), 5′Vβ8.1-TAGCGGCCGCCACCATGGACTCCTGGACCTTC (SEQ ID NO: 54), as well as constant chain-specific primers containing an EcoRI restriction site: 3′Cα-TGGAATTCCTAGCCTCTGGAATCCTTTCTC (SEQ ID NO: 55) and 3′Cβ2-TGGAAT TCCTAGCCTCTGGAATCCTTTCTC (SEQ ID NO: 56). The TCR genes were cloned separately as single TCR genes into the retroviral vector MP71-PRE (MP71-TCRα and MP71-TCRβ). GFP-encoding MP71 vector was used as a mock control.

Retroviral Transfer into PBMC

The TCR-containing retroviral vector plasmids pMP71G_{PRE} were cotransfected with plasmids harbouring retroviral proteins gag/pol (pcDNA3.1-murine leukemia virus (MLV)) and env (pAIF10A1-GALV) into 293T cells by calcium phosphate precipitation to generate amphotropic vector particles (35). PBMC activated for two days with IL-2 (50 U/mL) and OKT3 (50 ng/mL) were transduced twice with retrovirus-containing supernatant in 24-well non-tissue culture plates coated with RetroNectin (Takara, Apen, Germany) containing protamine sulfate (4 μg/mL) and IL-2 (100 U/mL). After addition of retroviral supernatant, the plates were spinoculated with 800×g for 1.5 h at 32° C. Medium was replaced by fresh medium after 48-72 h. Transduced PBMC were analyzed for multimer staining and surface markers as well as functional assays at different time points after transduction as indicated. Enrichment of transduced cells by multimer sorting was performed where indicated. PBMC transduced with a GFP-containing MP71 vector control were used as mock control.

Results:

HER2/Neu-Specific Allorestricted T Cell Clones Could be Isolated After Stimulation with HER2/Neu (369)-Peptide Pulsed T2 Cells

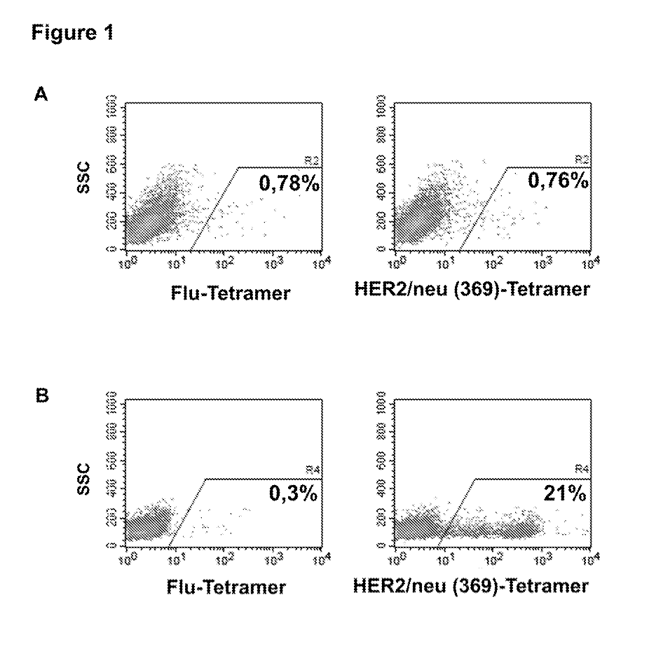

T2 cells were pulsed with the antigenic HER2/neu-derived peptide 369 (12) at different conditions (Table 1). We used peptide concentrations of either 10 μM or 0.1 μM for pulsing of T2 cells (Table 1). HLA-A2⁻ T cells from healthy donors were stimulated once or twice with peptide-pulsed T2 cells and subsequently FACS-sorted using multimers. Following stimulation the frequency of HER2/neu-positive cells was between 0.05 and 0.8% before sorting and did not show major differences between the different conditions. Frequencies of multimer-positive cells stained with the control multimer Flu were at a similar range (FIG. 1A). However, HER2/neu (369)-multimer-positive cells could be enriched by sorting with the specific multimer (Table 1, FIG. 1B).

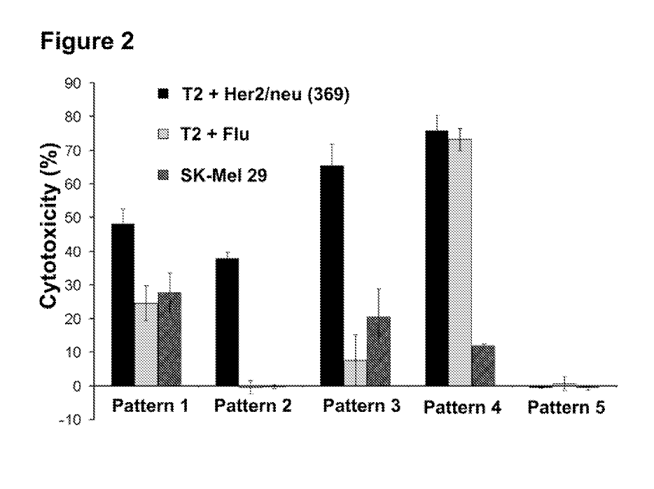

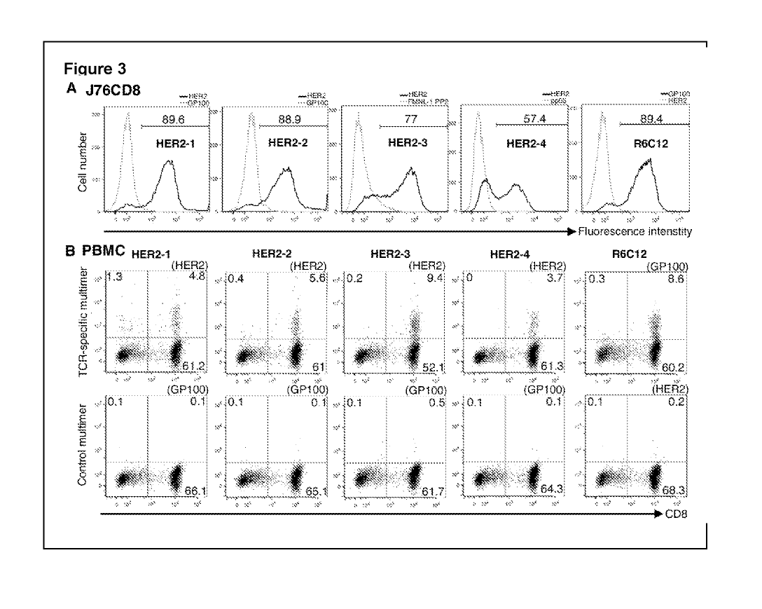

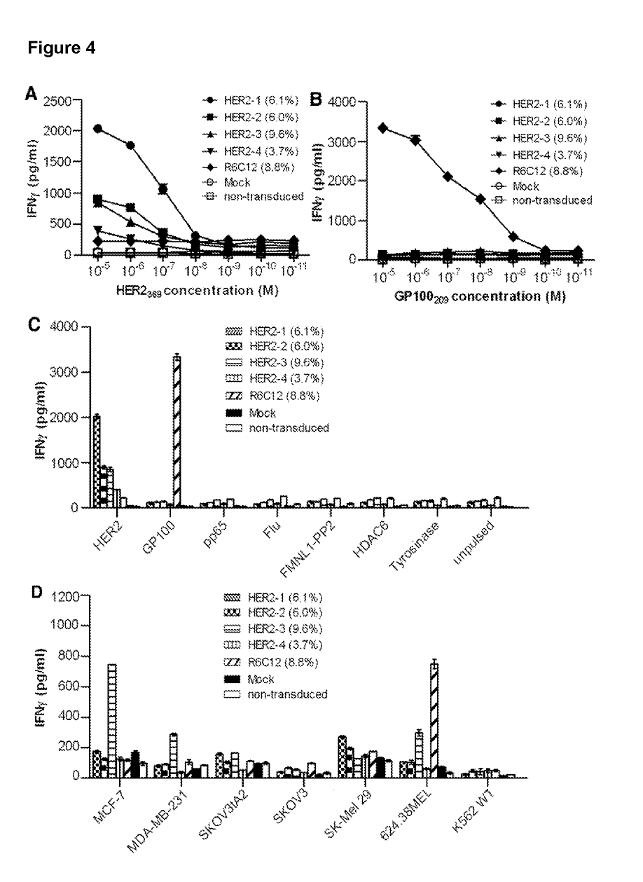

Sorted T-cell lines containing enriched HER2/neu-multimer positive cells were cloned by limiting dilutions and generated T-cell clones were investigated in a screening assay for peptidespecificity and tumorreactivity (FIG. 2, Table 2). Five different patterns in response to T2 cells pulsed with the HER2/neu (369)-peptide and the Flu control as well as HER2/neu-overexpressing tumor cells were detected (FIG. 2). Interestingly, most of the sorted lines and clones derived from condition I (1× stimulation with 10 μM HER2/neu (369)-peptide) showed the reaction pattern 1, demonstrating preferential recognition of T2 cells pulsed with HER2/neu (369) and tumorreactivity but also partial alloreactivity (FIG. 2, Table 2). In contrast, clones derived from conditions III and IV (2× stimulation with either 10 μM or 0.1 μM HER2/neu (369)-peptide) resulted in several clones reacting according to pattern 2 with high peptide specificity without cross-reactivity against the control peptide Flu (FIG. 2, Table 2). However, these T-cell clones were not reactive against tumor cells. These two conditions also resulted in many unspecific clones (patterns 4 and 5). Only one clone represented the favorable pattern 3 with high peptide specificity and tumor reactivity. This clone (D1/HER2-1) was derived from condition II (1× stimulation with 0.1 μM HER2/neu (369)-peptide). Extensive testing of most T cell clones, especially D1 (HER2-1), was not possible as they were mostly short-lived. Therefore, mRNA was isolated from these T cells for TCR analysis and subsequent TCR gene transfer.

TCR Repertoire of HER2/Neu-Specific HLA-A2-Allorestricted T-Cell Clones

TCR analysis of selected clones showed a diverse spectrum of different Vα chains (n=9) and Vβ chains (n=6) (Table 3). Identical CDR3-regions occurred only within the same original stimulation condition with the exception for one TCRα chain from condition I (clone B7) which was also present in clones derived from condition IV (G3 (HER2-2), G8, H3, H8). All clones obtained from condition IV (clones G3 (HER2-2), G8, H3, H8) had identical TCR. The TCR sequences from clone D1 (HER2-1) were unique.

Retroviral TCR Transfer of HER2₃₆₉-Specific HLA-A2-Allorestricted TCR into Recipient Cells Results in Positive HER2₃₆₉-Multimer Staining as Well as Peptide-Specific Function and Tumor Reactivity

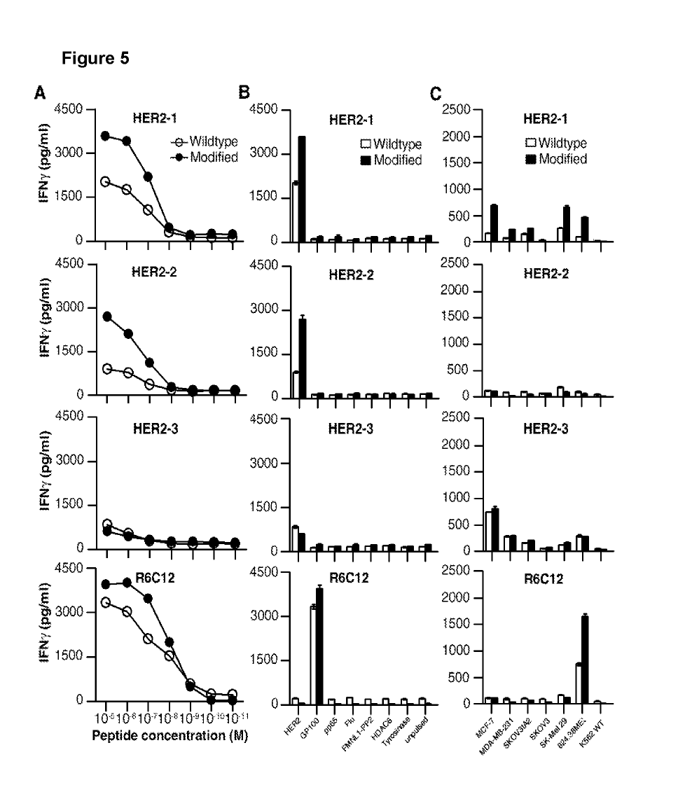

The TCR α and β chain genes derived from four different clones demonstrating high peptide-specificity were used for cloning of TCR chain genes for transfer studies. In addition, we used the GP100₂₀₉-specific TCR derived from clone R6C12 (friendly provided by R. Morgan), a CMV-pp65₄₉₅ specific TCR (JG-9) (friendly provided by A. Moosmann) and the FMNL1-PP2-specific TCR SK22 (26) as control TCR with defined specificities for other antigens than HER2. Retroviral TCR gene transfer with unmodified TCR α- and β-chain genes using single TCR chain vectors into TCR knock-out J76CD8 or PBMC resulted in cells positive for the specific MHC-peptide multimer but negative for control multimers (FIGS. 3A and B). TCR HER2-1 was the only HER2₃₆₉-specific TCR demonstrating a significant percentage of multimer-positive cells in the CD8⁻ population (FIG. 3B) corresponding to multimer-positivity of HER2-1-transduced J76 lacking CD8 (data not shown). PBMC transduced with unmodified TCR chain pairs from HER2₃₆₉-specific T-cell clones as well as the control TCR R6C12 exerted reactivity towards the specific peptide in a dose-dependent manner (FIGS. 4A and 4B). All transduced TCR demonstrated high peptide specificity and did not respond to a panel of irrelevant peptides (FIG. 4C). Analyzing the tumor reactivity of PBMC transduced with diverse HER2₃₆₉-specific TCR, we observed mainly tumor reactivity of HER2-3 against diverse tumor cell lines as MCF-7 and MDA-MB 231 as well as marginal tumor reactivity of HER2-1 against SK-Mel 29 (FIG. 4D).

TCR Modifications Improve Transgenic TCR Expression and Specific Functions of TCR-Transduced PBMC

In order to improve expression and reactivity of the three TCR with the highest HER2₃₆₉ reactivity, we introduced modifications into TCR constructs of HER2-1, HER2-2 and HER2-3. We first murinized constant chains as previously described. We additionally performed codon optimization and cloned both TCR β- and α-chain genes in one vector separated by the picorna virus derived peptide element P2A as previously described (35). These modifications improved multimer staining as well as peptide-specific function (FIGS. 5A and 5B) of TCR HER2-1 and HER2-2. Functional avidity of PBMC transduced with HER2-1 was increased by these modifications when compared to transduction of unmodified chain genes (FIG. 5A). In addition, these modifications improved tumor reactivity of HER2-1 but not HER2-2 and HER2-3 (FIG. 5C). Modification of TCR HER2-3 revealed only improvement of multimer staining but not HER2₃₆₉-specific function and tumor reactivity (FIG. 5A-C).

TABLE 1
In vitro conditions used for generation of
HER2/neu (369)-specific allo-HLA-A2-restricted T-cell lines

| | Number of | Peptide | Multimer-positive |
|---|---|---|---|
| | stimulations | (μM) | cells in sorted cell |
| | before | loading on | line before |
| Condition | sorting | T2 cells | cloning (%) |
| I | 1 | 10 | 65 |
| II | 1 | 0.01 | 17 |
| III | 2 | 10 | 21 |
| IV | 2 | 0.01 | 18 |

TABLE 2
Number of clones derived from different peptide stimulation conditions
representing the specific functional patterns shown in FIG. 2.

| | Pattern | Pattern | | | | |
|---|---|---|---|---|---|---|
| Condition | 1 | 2 | Pattern 3 | Pattern 4 | Pattern 5 | Total |
| I | 13 | — | — | 4 | 2 | 19 |
| II | — | — | 1 | — | 2 | 3 |
| III | 19 | 11 | — | 18 | 7 | 55 |
| IV | 1 | 4 | — | 8 | 8 | 21 |

TABLE 3
TCR alpha and beta CDR3 sequences
of selected T-cell clones derived from
different stimulation conditions

| | | TCR alpha chain sequences | | | |
|---|---|---|---|---|---|
| Condition | Clone | Vα | CDR3 | Jα | |
| I | A2 | 14.1 | CAYMEGNTDKLIFG | 34.1 | |
| I | A3 | 6.1 | CAMREGSSFGNEKTFG | 48.1 | |
| I | A3 | 19.1 | CAAEAPGGTSYGKLTFG | 52.1 | |
| I | B7 | 10.1 | CAGVPSNDYKLSFG | 20.1 | |
| I | B7 | 14.1 | CAYMEGNTDKLIFG | 34.1 | |
| I | B10 | 6.1 | CAMREGSSFGNEKTFG | 48.1 | |
| I | B10 | 7.1 | CAVVGGFKTIFG | 9.1 | |
| II | D1 | 12.1 | CALYTTDSWGKLQFG | 24.2 | |
| III | E1 | 23.1 | CAVRPQNDYKLSFG | 20.1 | |
| III | E2 | 18.1 | CAFGFGFGNVLHCG | 35.1 | |
| III | E2 | 23.1 | CAVRPQNDYKLSFG | 20.1 | |
| IV | G3 | 10.1 | CAGVPSNDYKLSFG | 20.1 | |
| IV | G8 | 10.1 | CAGVPSNDYKLSFG | 20.1 | |
| IV | H3 | 10.1 | CAGVPSNDYKLSFG | 20.1 | |
| IV | H5 | 10.1 | CAGVPSNDYKLSFG | 20.1 | |
| | | TCR beta chain sequences | | | |
| Condition | Clone | Vβ | CDR3 | Jβ | Cβ |
| I | A2 | 6.2 | CASSLDLIGLQETQYFG | 2.5 | 2 |
| I | A3 | 8.3 | CASGLGAGGPGDTQYFG | 2.3 | 2 |
| I | B7 | 6.2 | CASSLDLIGLQETQYFG | 2.5 | 2 |
| I | B10 | 8.3 | CASGLGAGGPGDTQYFG | 2.3 | 2 |
| II | D1 | 8.1 | CASSFVLGDTQYFG | 2.3 | 2 |
| III | E1 | 8.1 | CASSSWTSGDEQFFG | 2.1 | 2 |
| III | E2 | 8.1 | CASSSWTSGDEQFFG | 2.1 | 2 |
| III | E2 | 14 | CASSLSGQGPTNYGYTFG | 1.2 | 1 |
| IV | G3 | 8.1 | CASSPPLGSGIYEQYFG | 2.7 | 2 |
| IV | G8 | 8.1 | CASSPPLGSGIYEQYFG | 2.7 | 2 |
| IV | H3 | 8.1 | CASSPPLGSGIYEQYFG | 2.7 | 2 |
| IV | H5 | 8.1 | CASSPPLGSGIYEQYFG | 2.7 | 2 |

TABLE 4

| | TCR β-chain sequence | | | |
|---|---|---|---|---|
| | Vβ | CDR3 | Jβ | Cβ |
| 96 | Vβ6.2 | C A S S L A A D E Q Y F G | Jβ2.7 | Cβ2 |
| (HER2-3) | | tgt gcc agc agc tta gcg gcg gac gag cag tac ttc ggg | | |
| | Vβ4.1 | C S V R L E E K L F F G | Jβ1.4 | Cβ1 |
| | | tgc agc gtt cgt ttg gag gaa aaa ctg ttt ttt ggc | | |
| BS 2 | Vβ14.1 | C A S S L Y G A D Y E Q Y F G | Jβ2.7 | Cβ2 |
| | | tgt gcc agc agt tta tat ggg gcc gac tac gag cag tac ttc ggg | | |
| BS 3 | Vβ13.2 | C A S S Q W D S N Q P Q H F G | Jβ1.5 | Cβ1 |
| | | tgt gcc agc agt caa tgg gat agc aat cag ccc cag cat ttt ggt | | |
| A2 | Vβ 6.2 | C A S S L D L I G L Q E T Q Y F G | Jβ2.5 | Cβ2 |
| | | tgt gcc agc agc tta gac tta att ggc cta caa gag acc cag tac ttc ggg | | |
| A3 | Vβ8.3 | C A S G L G A G G P G D T Q Y F G | Jβ2.3 | Cβ2 |
| | | tgt gct agt ggt tta ggg gcg gga ggg ccc gga gat act cag tat ttt ggc | | |
| D1 | Vβ8.1 | C A S S F V L G D T Q Y F G | Jβ2.3 | Cβ2 |
| (HER2-1) | | tgt gcc agc agt ttc gtg ctt gga gat acg cag tat ttt ggc | | |
| G3 | Vβ8.1 | C A S S P P L G S G I Y E Q Y F G | Jβ2.7 | Cβ2 |
| (HER2-2) | | tgt gcc agc agt cca cca ctc ggc agc ggg att tac gag cag tac ttc ggg | | |
| E1 | Vβ8.1 | C A S S S W T S G D E Q F F G | Jβ2.1 | CB2 |
| (HER2-4) | | tgt gcc agc agt tca tgg act agg ggg gat gag cag ttc ttc ggg | | |
| E2 | Vβ14 | C A S S L S G Q G P T N Y G Y T F G | Jβ1.2 | Cβ1 |
| | | tgt gcc agc agt tta agc gga cag ggg cca aca aac tat ggc tac acc ttc ggt | | |

| | TCR α-chain sequence | | |
|---|---|---|---|
| | Vα | CDR3 | Jα |
| 96 Well | Vα 1.1 | C A V N P N D Y K L S F G | Jα 20.1 |
| (HER2-3) | | tgt gcc gtg aac cct aac gac tac aag ctc agc ttt gga | |
| | Vα 14.2 | C A F I D S G A G S Y Q L T F G | Jα 28.1 |
| | | tgt gct ttc att gac tct ggg gct ggg agt tac caa ctc act ttc ggg | |
| BS 1 | Vα 6.1 | C A M R V S G A G S Y Q L T F G | Jα 28.1 |
| | | tgt gca atg agg gta tct ggg gct ggg agt tac caa ctc act ttc ggg | |
| BS 2 | Vα 2.1 | C A V T N S G G Y Q K V T F G | Jα 13.1 |
| | | tgt ggc gtg acc aat tct ggg ggt tac cag aaa gtt acc ttt gga | |
| BS 3 | Vα 2.1 | C A V N G G G A D G L T F G | Jα 45.1 |
| | | tgt gcc gtg aac ggc gga ggt gct gac gga ctc acc ttt ggc | |
| A2 | Vα 14.1 | C A Y M E G N T D K L I F G | Jα 34.1 |
| | | tgt gct tat atg gag ggg aac acc gac aag ctc atc ttt ggg | |
| A3 | Vα 6.1 | C A M R E G S S F G N E K L T F G | Jα 48.1 |
| | | tgt gca atg aga gag ggc tct tcc ttt gga aat gag aaa tta acc ttt ggg | |
| | Vα 19.1 | C A A E A P G G T S Y G K L T F G | Jα 52.1 |
| | | tgt gct gcc gag gcc cct ggt ggt act agc tat gga aag ctg aca ttt gga | |
| B10 | Vα 7.1 | C A V V G G F K T I F G | Jα 9.1 |
| | | tgc gct gtg gtt gga ggc ttc aaa act atc ttt gga | |
| D1 | Vα 12.1 | C A L Y T T D S W G K L Q F G | Jα 24.2 |
| (HER2-1) | | tgt gct ctt tat aca act gac agc tgg ggg aaa ttg cag ttt gga | |
| G3 | Vα 10.1 | C A G V P S N D Y K L S F G | Jα 20.1 |
| (HER2-2) | | tgt gca gga gtc ccc tct aac gac tac aag ctc agc ttt gga | |
| E1 | Vα 23.1 | C A V R P Q N D Y K L S F G | Jα 20.1 |
| (HER2-4) | | tgt gct gtg agg ccc cag aac gac tac aag ctc agc ttt gga | |
| E2 | Vα 18.1 | C A F G F G F G N V L H C G | Jα 35.1 |
| | | tgt gcc ttt ggc ttc ggc ttt ggg aat gtg ctg cat tgc ggg | |

TABLE 5
Peptide and tumor recognition of PBMC transduced with HER2-2α in
combination with different TCR β-chains

| | HER2-2 | | | | | |
|---|---|---|---|---|---|---|
| α chain | AV27 | | | | | |
| | HER2-1 | HER2-2 | HER2-3 | HER2-4 | | Non- |
| β chain | BV12 | BV12 | BV7 | BV12 | Mock | transduced |
| HER2₃₆₉ | 10.5* | 16.2* | 0.2* | 10.8 | | |
| multimer⁺ | | | | | | |
| (%) in CD8⁺ | | | | | | |
| T2 + 10⁻⁵ M | 99 | 2697 | 74 | 3624 | 48 | 33 |
| HER2₃₆₉ | | | | | | |
| T2 + 10⁻⁶ M | 86 | 2094 | 57 | 3924 | 42 | 39 |
| HER2₃₆₉ | | | | | | |
| T2 + 10⁻⁷ M | 53 | 1114 | 52 | 3261 | 41 | 37 |
| HER2₃₆₉ | | | | | | |
| T2 + 10⁻⁸ M | 60 | 276 | 45 | 2129 | 61 | 30 |
| HER2₃₆₉ | | | | | | |
| T2 + 10⁻⁹ M | 35 | 175 | 53 | 1208 | 47 | 20 |
| HER2₃₆₉ | | | | | | |
| T2 + 10⁻¹⁰ M | 36 | 158 | 38 | 826 | 28 | 28 |
| HER2₃₆₉ | | | | | | |
| T2 + 10⁻¹¹ M | 70 | 147 | 42 | 754 | 31 | 24 |
| HER2₃₆₉ | | | | | | |
| T2 unpulsed | 32 | 183 | 44 | 820 | 35 | 25 |

TABLE 6
Peptide and tumor recognition of PBMC transduced with HER2-2α
in combination with different TCR β-chains followed by
depletion of CD8⁺ cells

| α chain | HER2-2 AV27 | | |
|---|---|---|---|
| β chain | HER2-4 BV12 | Mock | Non-transduced |
| HER2₃₆₉ multimer⁺ (%) | 0.1 | | |
| in CD8⁺ | | | |
| HER2₃₆₉ muitimer⁺ (%) | 14.1 | | |
| in CD8⁻ | | | |
| T2 + 10⁻⁵M HER2₃₆₉ | 4287 | 27 | 30 |
| T2 + 10⁻⁶M HER2₃₆₉ | 4347 | 24 | 19 |
| T2 + 10⁻⁷M HER2₃₆₉ | 4059 | 16 | 22 |
| T2 + 10⁻⁸M HER2₃₆₉ | 3827 | 14 | 16 |
| T2 + 10⁻⁹M HER2₃₆₉ | 1806 | 11 | 31 |
| T2 + 10⁻¹⁰M HER2₃₆₉ | 750 | 11 | 26 |
| T2 unpulsed | 602 | 17 | 15 |

*Indicates usage of TCR construct modified by codon optimization, usage of murinized constant chains and bicistronic vectors

### LITERATURE

1. Slavin, S. Immunotherapy of cancer with alloreactive lymphocytes. Lancet Oncol, 2: 491-498, 2001.
2. Blaise, D., Bay, J. O., Faucher, C., Michallet, M., Boiron, J. M., Choufi, B., Cahn, J. Y., Gratecos, N., Sotto, J. J., Francois, S., Fleury, J., Mohty, M., Chabannon, C., Bilger, K., Gravis, G., Viret, F., Braud, A. C., Bardou, V. J., Maraninchi, D., and Viens, P. Reduced-intensity preparative regimen and allogeneic stem cell transplantation for advanced solid tumors. Blood, 103: 435-441, 2004.
3. Childs, R., Chernoff, A., Contentin, N., Bahceci, E., Schrump, D., Leitman, S., Read, E. J., Tisdale, J., Dunbar, C., Linehan, W. M., Young, N. S., and Barrett, A. J. Regression of metastatic renal-cell carcinoma after nonmyeloablative allogeneic peripheral-blood stem-cell transplantation. N Engl J Med, 343: 750-758, 2000.
4. Bishop, M. R., Fowler, D. H., Marchigiani, D., Castro, K., Kasten-Sportes, C., Steinberg, S. M., Gea-Banacloche, J. C., Dean, R., Chow, C. K., Carter, C., Read, E. J., Leitman, S., and Gress, R. Allogeneic lymphocytes induce tumor regression of advanced metastatic breast cancer. J Clin Oncol, 22: 3886-3892, 2004.
5. Carella, A. M., Beltrami, G., Corsetti, M. T., Nati, S., Musto, P., Scalzulli, P., Gonella, R., Ballestrero, A., and Patrone, F. Reduced intensity conditioning for allograft after cytoreductive autograft in metastatic breast cancer. Lancet, 366: 318-320, 2005.
6. Morris, E. C., Bendle, G. M., and Stauss, H. J. Prospects for immunotherapy of malignant disease. Clin Exp Immunol, 131: 1-7, 2003.
7. Moris, A., Teichgraber, V., Gauthier, L., Buhring, H. J., and Rammensee, H. G. Cutting edge: characterization of allorestricted and peptide-selective alloreactive T cells using HLA-tetramer selection. J Immunol, 166: 4818-4821, 2001.
8. Stanislawski, T., Voss, R. H., Lotz, C., Sadovnikova, E., Willemsen, R. A., Kuball, J., Ruppert, T., Bolhuis, R. L., Melief, C. J., Huber, C., Stauss, H. J., and Theobald, M. Circumventing tolerance to a human MDM2-derived tumor antigen by TCR gene transfer. Nat Immunol, 2: 962-970, 2001.
9. Xue, S. A., Gao, L., Hart, D., Gillmore, R., Qasim, W., Thrasher, A., Apperley, J., Engels, B., Uckert, W., Morris, E., and Stauss, H. Elimination of human leukemia cells in NOD/SCID mice by WT1-TCR gene-transduced human T cells. Blood, 106: 3062-3067, 2005.
10. Morgan, R. A., Dudley, M. E., Wunderlich, J. R., Hughes, M. S., Yang, J. C., Sherry, R. M., Royal, R. E., Topalian, S. L., Kammula, U. S., Restifo, N. P., Zheng, Z., Nahvi, A., de Vries, C. R., Rogers-Freezer, L. J., Mavroukakis, S. A., and Rosenberg, S. A. Cancer regression in patients after transfer of genetically engineered lymphocytes. Science, 314: 126-129, 2006.
11. de Witte, M. A., Coccoris, M., Wolkers, M. C., van den Boom, M. D., Mesman, E. M., Song, J. Y., van der Valk, M., Haanen, J. B., and Schumacher, T. N. Targeting self-antigens through allogeneic TCR gene transfer. Blood, 108: 870-877, 2006.
12. Fisk, B., Blevins, T. L., Wharton, J. T., and Ioannides, C. G. Identification of an immunodominant peptide of HER-2/neu protooncogene recognized by ovarian tumor-specific cytotoxic T lymphocyte lines. J Exp Med, 181: 2109-2117, 1995.
13. Coussens, L., Yang-Feng, T. L., Liao, Y. C., Chen, E., Gray, A., McGrath, J., Seeburg, P. H., Libermann, T. A., Schlessinger, J., Francke, U., and et al. Tyrosine kinase receptor with extensive homology to EGF receptor shares chromosomal location with neu oncogene. Science, 230: 1132-1139, 1985.
14. Bargmann, C. I., Hung, M. C., and Weinberg, R. A. The neu oncogene encodes an epidermal growth factor receptor-related protein. Nature, 319: 226-230, 1986.
15. Chazin, V. R., Kaleko, M., Miller, A. D., and Slamon, D. J. Transformation mediated by the human HER-2 gene independent of the epidermal growth factor receptor. Oncogene, 7: 1859-1866, 1992.
16. Slamon, D. J., Clark, G. M., Wong, S. G., Levin, W. J., Ullrich, A., and McGuire, W. L. Human breast cancer: correlation of relapse and survival with amplification of the HER-2/neu oncogene. Science, 235: 177-182, 1987.
17. Cobleigh, M. A., Vogel, C. L., Tripathy, D., Robert, N. J., Scholl, S., Fehrenbacher, L., Wolter, J. M., Paton, V., Shak, S., Lieberman, G., and Slamon, D. J. Multinational study of the efficacy and safety of humanized anti-HER2 monoclonal antibody in women who have HER2-overexpressing metastatic breast cancer that has progressed after chemotherapy for metastatic disease. J Clin Oncol, 17: 2639-2648, 1999.
18. Slamon, D. J., Leyland-Jones, B., Shak, S., Fuchs, H., Paton, V., Bajamonde, A., Fleming, T., Eiermann, W., Wolter, J., Pegram, M., Baselga, J., and Norton, L. Use of chemotherapy plus a monoclonal antibody against HER2 for metastatic breast cancer that overexpresses HER2. N Engl J Med, 344: 783-792, 2001.
19. Peoples, G. E., Goedegebuure, P. S., Smith, R., Linehan, D. C., Yoshino, I., and Eberlein, T. J. Breast and ovarian cancer-specific cytotoxic T lymphocytes recognize the same HER2/neu-derived peptide. Proc Natl Acad Sci U S A, 92: 432-436, 1995.
20. Rongcun, Y., Salazar-Onfray, F., Charo, J., Malmberg, K. J., Evrin, K., Maes, H., Kono, K., Hising, C., Petersson, M., Larsson, 0., Lan, L., Appella, E., Sette, A., Celis, E., and Kiessling, R. Identification of new HER2/neu-derived peptide epitopes that can elicit specific CTL against autologous and allogeneic carcinomas and melanomas. J Immunol, 163: 1037-1044, 1999.
21. Zaks, T. Z. and Rosenberg, S. A. Immunization with a peptide epitope (p369-3T7) from HER-2/neu leads to peptide-specific cytotoxic T lymphocytes that fail to recognize HER-2/neu+ tumors. Cancer Res, 58: 4902-4908, 1998.
22. Knutson, K. L., Schiffman, K., Cheever, M. A., and Disis, M. L. Immunization of cancer patients with a HER-2/neu, HLA-A2 peptide, p369-3T7, results in short-lived peptide-specific immunity. Clin Cancer Res, 8: 1014-1018, 2002.
23. Disis, M. L., Gooley, T. A., Rinn, K., Davis, D., Piepkorn, M., Cheever, M. A., Knutson, K. L., and Schiffman, K. Generation of T-cell immunity to the HER-2/neu protein after active immunization with HER-2/neu peptide-based vaccines. J Clin Oncol, 20: 2624-2632, 2002.
24. Peoples, G. E., Gurney, J. M., Hueman, M. T., Woll, M. M., Ryan, G. B., Stoner, C. E., Fisher, C., Shriver, C. D., Ioannides, C. G., and Ponniah, S. Clinical trial results of a HER2/neu (E75) vaccine to prevent recurrence in high-risk breast cancer patients. J Clin Oncol, 23: 7536-7545, 2005.
25. Bernhard, H., Neudorfer, J., Gebhard, K., Conrad, H., Hermann, C., Nahrig, J., Fend, F., Weber, W., Busch, D. H., and Peschel, C. Adoptive transfer of autologous, HER2-specific, cytotoxic T lymphocytes for the treatment of HER2-overexpressing breast cancer. Cancer Immunol Immunother, 57: 271-280, 2008.
26. Schuster, I. G., Busch, D. H., Eppinger, E., Kremmer, E., Milosevic, S., Hennard, C., Kuttler, C., Ellwart, J. W., Frankenberger, B., Noessner, E., Salat, C., Bogner, C., Borkhardt, A., Kolb, H. J., and Krackhardt, A. M. Allorestricted T cells with specificity for the FMNL1-derived peptide PP2 have potent antitumor activity against hematological and other malignancies. Blood, 2007.
27. Busch, D. H., Pilip, I. M., Vijh, S., and Pamer, E. G. Coordinate regulation of complex T cell populations responding to bacterial infection. Immunity, 8: 353-362, 1998.
28. Knabel, M., Franz, T. J., Schiemann, M., Wulf, A., Villmow, B., Schmidt, B., Bernhard, H., Wagner, H., and Busch, D. H. Reversible MHC multimer staining for functional isolation of T-cell populations and effective adoptive transfer. Nat Med, 8: 631-637, 2002.
29. Neudorfer, J., Schmidt, B., Huster, K. M., Anderl, F., Schiemann, M., Holzapfel, G., Schmidt, T., Germeroth, L., Wagner, H., Peschel, C., Busch, D. H., and Bernhard, H. Reversible HLA multimers (Streptamers) for the isolation of human cytotoxic T lymphocytes functionally active against tumor- and virus-derived antigens. J Immunol Methods, 320: 119-131, 2007.
30. Savage, P. A., Boniface, J. J., and Davis, M. M. A kinetic basis for T cell receptor repertoire selection during an immune response. Immunity, 10: 485-492, 1999.
31. Krackhardt, A. M., Witzens, M., Harig, S., Hodi, F. S., Zauls, A. J., Chessia, M., Barrett, P., and Gribben, J. G. Identification of tumor-associated antigens in chronic lymphocytic leukemia by SEREX. Blood, 100: 2123-2131, 2002.
32. Roth, M. E., Lacy, M. J., McNeil, L. K., and Kranz, D. M. Analysis of T cell receptor transcripts using the polymerase chain reaction. Biotechniques, 7: 746-754, 1989.
33. Nomenclature for T-cell receptor (TCR) gene segments of the immune system. WHO-IUIS Nomenclature Sub-Committee on TCR Designation. Immunogenetics, 42: 451-453, 1995.
34. Engels, B., Noessner, E., Frankenberger, B., Blankenstein, T., Schendel, D. J., and Uckert, W. Redirecting human T lymphocytes toward renal cell carcinoma specificity by retroviral transfer of T cell receptor genes. Hum Gene Ther, 16: 799-810, 2005.
35. Leisegang, M., Engels, B., Meyerhuber, P., Kieback, E., Sommermeyer, D., Xue, S. A., Reu13, S., Stauss, H., and Uckert, W. Enhanced functionality of T cell receptor-redirected T cells is defined by the transgene cassette. J Mol Med, in press 2008.
36. Dudley, M. E., Wunderlich, J. R., Robbins, P. F., Yang, J. C., Hwu, P., Schwartzentruber, D. J., Topalian, S. L., Sherry, R., Restifo, N. P., Hubicki, A. M., Robinson, M. R., Raffeld, M., Duray, P., Seipp, C. A., Rogers-Freezer, L., Morton, K. E., Mavroukakis, S. A., White, D. E., and Rosenberg, S. A. Cancer regression and autoimmunity in patients after clonal repopulation with antitumor lymphocytes. Science, 298: 850-854, 2002.
37. Dudley, M. E., Wunderlich, J. R., Yang, J. C., Sherry, R. M., Topalian, S. L., Restifo, N. P., Royal, R. E., Kammula, U., White, D. E., Mavroukakis, S. A., Rogers, L. J., Gracia, G. J., Jones, S. A., Mangiameli, D. P., Pelletier, M. M., Gea-Banacloche, J., Robinson, M. R., Berman, D. M., Filie, A. C., Abati, A., and Rosenberg, S. A. Adoptive cell transfer therapy following non-myeloablative but lymphodepleting chemotherapy for the treatment of patients with refractory metastatic melanoma. J Clin Oncol, 23: 2346-2357, 2005.
38. Vanclee, A., van Gelder, M., Schouten, H. C., and Bos, G. M. Graft-versus-tumor effects on murine mammary carcinoma in a model of nonmyeloablative haploidentical stem cell transplantation. Bone Marrow Transplant, 37: 1043-1049, 2006.

1. A T cell receptor (TCR) recognizing HER2/neu derived peptide 369 and capable of inducing peptide specific killing of a target cell overexpressing HER2/neu, wherein the TCR specifically recognizes the peptide of SEQ ID NO: 1.

2. The TCR of claim 1, which contains or consists of one of the amino acids of the TCR alpha chains of SEQ ID NO: 2-14 and/or one of the amino acids of the TCR beta chains of SEQ ID NO: 15-24.

3. The TCR of claim 2, which contains or consists of the amino acids of the TCR alpha chain of SEQ ID NO: 2 or 5.

4. The TCR of claim 2 or 3, which contains the alpha chain of SEQ ID NO: 5 and the beta chain of SEQ ID NO: 23.

5. An antigen specific T cell, comprising a TCR as defined in claim 1.

6. The T cell of claim 5, wherein the T cell is a T cell with effector cell characteristics.

7. The T cell of claim 6, which is an autologous or allogeneic T cell.

8. A nucleic acid coding for a TCR as defined in claim 1 or 2 or comprising or consisting of one of SEQ ID NO: 25-47.

9. A vector or mRNA, which comprises the nucleic acid of claim 8.

10. The vector of claim 9, which is a plasmid or a retroviral vector.

11. A cell which has been transformed with the vector or mRNA of claim 9 or 10.

12. A pharmaceutical composition, which comprises the T cells of claims 6 or the cell of claim 11 and a pharmaceutically acceptable carrier.

13. The pharmaceutical composition of claim 12, which is an infusion, injection or a vaccine.

14. The pharmaceutical composition of claim 12 for use in the treatment of tumors characterized by overexpression of HER2/neu.

15. (canceled)

16. A method of generating antigen specific T cells comprising the steps of
a) providing the HER2/neu derived antigenic peptide 369;
b) pulsing T2 cells with said peptide;
c) stimulating T cells with the peptide pulsed T2 cells;
d) selecting those T cells which are specific for the HER2/neu derived antigenic peptide.

17. The T cell of claim 6, which is a cytokine producing T cell, a cytotoxic T cell or regulatory T cells, preferably CD4+ or CD8+ T cells.

18. The cell of claim 11, which is a PBMC.

19. The pharmaceutical composition of claim 14, for use in the treatment of breast cancer.

20. A method for treating cancer in a subject comprising administering a therapeutically effective amount of the pharmaceutical composition of claim 12 to the subject.

21. The method of claim 20, wherein the cancer is breast cancer.


SEQUENCE LISTING (56 sequences)

SEQ ID NO: 1 - protein, 9 aa, Artificial
  peptide 369 of Her2/neu
KIFGSLAFL

SEQ ID NO: 2 - protein, 15 aa, Artificial
  TCR alpha chain sequence D1; Her 2-1 alpha
CALYTTDSWGKLQFG

SEQ ID NO: 3 - protein, 16 aa, Artificial
  TCR alpha chain sequence A3
CAMREGSSFGNEKTFG

SEQ ID NO: 4 - protein, 17 aa, Artificial
  TCR alpha chain sequence A3
CAAEAPGGTSYGKLTFG

SEQ ID NO: 5 - protein, 14 aa, Artificial
  TCR alpha chain sequence G3; Her 2-2 alpha
CAGVPSNDYKLSFG

SEQ ID NO: 6 - protein, 12 aa, Artificial
  TCR alpha chain sequence B10
CAVVGGFKTIFG

SEQ ID NO: 7 - protein, 14 aa, Artificial
  TCR alpha chain sequence A2
CAYMEGNTDKLIFG

SEQ ID NO: 8 - protein, 14 aa, Artificial
  TCR alpha chain sequence E1; Her 2-4 alpha
CAVRPQNDYKLSFG

SEQ ID NO: 9 - protein, 14 aa, Artificial
  TCR alpha chain sequence E2
CAFGFGFGNVLHCG

SEQ ID NO: 10 - protein, 13 aa, Artificial
  TCR alpha chain sequence 96 well; Her 2-3 alpha
CAVNPNDYKLSFG

SEQ ID NO: 11 - protein, 16 aa, Artificial
  TCR alpha chain sequence 96 well
CAFIDSGAGSYQLTFG

SEQ ID NO: 12 - protein, 16 aa, Artificial
  TCR alpha chain sequence BS 1
CAMRVSGAGSYQLTFG

SEQ ID NO: 13 - protein, 15 aa, Artificial
  TCR alpha chain sequence  BS 2
CAVTNSGGYQKVTFG

SEQ ID NO: 14 - protein, 14 aa, Artificial
  TCR alpha chain sequence BS 3
CAVNGGGADGLTFG

SEQ ID NO: 15 - protein, 13 aa, Artificial
  TCR beta chain sequence 96; Her 2-3 beta
CASSLAADEQYFG

SEQ ID NO: 16 - protein, 12 aa, Artificial
  TCR beta chain sequence 96
CSVRLEEKLFFG

SEQ ID NO: 17 - protein, 15 aa, Artificial
  TCR beta chain sequence BS 2
CASSLYGADYEQYFG

SEQ ID NO: 18 - protein, 15 aa, Artificial
  TCR beta chain sequence BS 3
CASSQWDSNQPQHFG

SEQ ID NO: 19 - protein, 17 aa, Artificial
  TCR beta chain sequence A2
CASSLDLIGLQETQYFG

SEQ ID NO: 20 - protein, 17 aa, Artificial
  TCR beta chain sequence A3
CASGLGAGGPGDTQYFG

SEQ ID NO: 21 - protein, 14 aa, Artificial
  TCR beta chain sequence D1; Her 2-1 beta
CASSFVLGDTQYFG

SEQ ID NO: 22 - protein, 17 aa, Artificial
  TCR beta chain sequence G3; Her 2-2 beta
CASSPPLGSGIYEQYFG

SEQ ID NO: 23 - protein, 15 aa, Artificial
  TCR beta chain sequence E1; Her 2-4 beta
CASSSWTSGDEQFFG

SEQ ID NO: 24 - protein, 18 aa, Artificial
  TCR beta chain sequence E2
CASSLSGQGPTNYGYTFG

SEQ ID NO: 25 - DNA, 42 bp, Artificial
  TCR alpha chain sequence A2
tgtgcttatatggaggggaacaccgacaagctcatctttggg

SEQ ID NO: 26 - DNA, 51 bp, Artificial
  TCR alpha chain sequence A3
tgtgcaatgagagagggctcttcctttggaaatgagaaattaacctttggg

SEQ ID NO: 27 - DNA, 51 bp, Artificial
  TCR alpha chain sequence A3
tgtgctgccgaggcccctggtggtactagctatggaaagctgacatttgga

SEQ ID NO: 28 - DNA, 36 bp, Artificial
  TCR alpha chain sequence B10
tgcgctgtggttggaggcttcaaaactatctttgga

SEQ ID NO: 29 - DNA, 45 bp, Artificial
  TCR alpha chain sequence D1
tgtgctctttatacaactgacagctgggggaaattgcagtttgga

SEQ ID NO: 30 - DNA, 42 bp, Artificial
  TCR alpha chain sequence G3
tgtgcaggagtcccctctaacgactacaagctcagctttgga

SEQ ID NO: 31 - DNA, 42 bp, Artificial
  TCR alpha chain sequence E1
tgtgctgtgaggccccagaacgactacaagctcagctttgga

SEQ ID NO: 32 - DNA, 42 bp, Artificial
  TCR alpha chain sequence E2
tgtgcctttggcttcggctttgggaatgtgctgcattgcggg

SEQ ID NO: 33 - DNA, 39 bp, Artificial
  TCR alpha chain sequence 96 well
tgtgccgtgaaccctaacgactacaagctcagctttgga

SEQ ID NO: 34 - DNA, 48 bp, Artificial
  TCR alpha chain sequence 96 well
tgtgctttcattgactctggggctgggagttaccaactcactttcggg

SEQ ID NO: 35 - DNA, 48 bp, Artificial
  TCR alpha chain sequence BS 1
tgtgcaatgagggtatctggggctgggagttaccaactcactttcggg

SEQ ID NO: 36 - DNA, 45 bp, Artificial
  TCR alpha chain sequence BS2
tgtggcgtgaccaattctgggggttaccagaaagttacctttgga

SEQ ID NO: 37 - DNA, 42 bp, Artificial
  TCR alpha chain sequence BS 3
tgtgccgtgaacggcggaggtgctgacggactcacctttggc

SEQ ID NO: 38 - DNA, 39 bp, Artificial
  TCR beta chain sequence 96
tgtgccagcagcttagcggcggacgagcagtacttcggg

SEQ ID NO: 39 - DNA, 36 bp, Artificial
  TCR beta chain sequence 96
tgcagcgttcgtttggaggaaaaactgttttttggc

SEQ ID NO: 40 - DNA, 45 bp, Artificial
  TCR beta chain sequence  BS 2
tgtgccagcagtttatatggggccgactacgagcagtacttcggg

SEQ ID NO: 41 - DNA, 45 bp, Artificial
  TCR beta chain sequence BS 3
tgtgccagcagtcaatgggatagcaatcagccccagcattttggt

SEQ ID NO: 42 - DNA, 51 bp, Artificial
  TCR beta chain sequence A2
tgtgccagcagcttagacttaattggcctacaagagacccagtacttcggg

SEQ ID NO: 43 - DNA, 51 bp, Artificial
  TCR beta chain sequence A3
tgtgctagtggtttaggggcgggagggcccggagatactcagtattttggc

SEQ ID NO: 44 - DNA, 42 bp, Artificial
  TCR beta chain sequence D1
tgtgccagcagtttcgtgcttggagatacgcagtattttggc

SEQ ID NO: 45 - DNA, 51 bp, Artificial
  TCR beta chain sequence  G3
tgtgccagcagtccaccactcggcagcgggatttacgagcagtacttcggg

SEQ ID NO: 46 - DNA, 45 bp, Artificial
  TCR beta chain sequence E1
tgtgccagcagttcatggactaggggggatgagcagttcttcggg

SEQ ID NO: 47 - DNA, 54 bp, Artificial
  TCR beta chain sequence E2
tgtgccagcagtttaagcggacaggggccaacaaactatggctacaccttcggt

SEQ ID NO: 48 - protein, 9 aa, artificial
  peptide MP58
GILGFVFTL

SEQ ID NO: 49 - protein, 9 aa, artificial
  tyrosinase derived peptide 369
YMNGTMSQV

SEQ ID NO: 50 - protein, 9 aa, artificial
  peptide PP2
RLPERMTTL

SEQ ID NO: 51 - protein, 9 aa, artificial
  HDAC6 derived peptide
RLAERMTTR

SEQ ID NO: 52 - DNA, 32 bp, artificial
  oligonucleotide
tagcggccgccaccatgctgactgccagcctg

SEQ ID NO: 53 - DNA, 32 bp, artificial
  oligonucleotide
tagcggccgccaccatggtcctgaaattctcc

SEQ ID NO: 54 - DNA, 32 bp, artificial
  oligonucleotide
tagcggccgccaccatggactcctggaccttc

SEQ ID NO: 55 - DNA, 30 bp, artificial
  oligonucleotide
tggaattcctagcctctggaatcctttctc

SEQ ID NO: 56 - DNA, 30 bp, artificial; identical to SEQ ID NO: 55
  oligonucleotide